(19) United States
(12) Patent Application Publication
(10) Pub. No.: US 2025/0025127 A1
(43) Pub. Date: Jan. 23, 2025

(54) ULTRASOUND IMAGING AND BACKSCATTER SYSTEM AND METHOD

(71) Applicant: SoundCath, Inc., Canoga Park, CA (US)

(72) Inventors: Donald Masters, Sylmar, CA (US); Eric Stoppenhagen, Santa Monica, CA (US)

(73) Assignee: SoundCath, Inc., Canoga Park, CA (US)

(21) Appl. No.: 18/353,604

(22) Filed: Jul. 17, 2023

(51) Int. Cl. A61B 8/12; A61B 8/00; A61B 8/08; B06B 1/06

(52) U.S. Cl. CPC A61B 8/12; A61B 8/0883; A61B 8/4488; B06B 1/0622; B06B 2201/76

(57) ABSTRACT

An ultrasound imaging and backscatter system comprises an ICE ultrasonic catheter having an ultrasonic pMUT transducer array disposed within a distal end of the ultrasonic catheter, and comprises a plurality of transducer array elements arranged on a substrate. A catheter shaft is connected at one end to a handle assembly and at other end to the ultrasonic transducer array. The catheter shaft houses an electronic flex cable which is in communication with a signal trace, and directs each of the plurality of transducer array elements to transmit and receive ultrasound beams, receive at least one signal from the plurality of transducer array elements, and construct at least one image characterizing one or more tissue component of a scanned object. The ultrasound imaging system is used for measuring tissue thickness, tissue scarring and lesion assessment for left and right atrium and ventricle tissue wall characterization and other intracardiac tissue abnormalities.

### FIELD OF THE DISCLOSURE

The present disclosure relates generally to the field of ultrasonic imaging systems. More particularly, some embodiments relate to piezoelectric transducer (PZT), Piezoelectric Micromachined Ultrasonic Transducer (pMUT) or micro-electromechanical (MEMS) ultrasonic catheters connected to an ultrasound imaging and backscatter system combined with mapping, ablation or other Intracardiac device for measuring tissue thickness, tissue scarring and lesion assessment for left and right atrium and ventricle tissue wall characterization and other intracardiac tissue abnormalities.

### BACKGROUND OF THE DISCLOSURE

Cardiovascular disease remains a leading cause of death in industrialized nations, with ischemic heart disease (IHD) and its sequelae being significant contributors. Over the years, noninvasive imaging techniques have played a crucial role in the detection, risk stratification, and management of patients with IHD. However, existing technologies have certain limitations that hinder their effectiveness in accurately assessing myocardial structure and composition.

Myocardial ischemia, caused by an imbalance between oxygen supply and consumption, initiates a series of pathological changes referred to as the ischemic cascade. Existing imaging methods, though valuable, have limitations in visualizing the early ultrastructural changes in the myocardium that occur as early as 10 to 15 minutes after the onset of ischemia. Moreover, the ability to accurately identify and quantify cytogenic and vasogenic edema, which are nonspecific responses to acute injury, remains limited.

Complications arising from unresolved ischemia, such as severe arrhythmias, cardiogenic shock, and myocardial rupture, necessitate the development of improved imaging techniques to enhance diagnosis and management. Additionally, there is a need for noninvasive methods that can effectively assess the chronic stage of ischemic heart disease, which is a major factor contributing to congestive heart failure. A demand arises for advancements in imaging technology to address these limitations and provide more comprehensive information about myocardial structure and composition.

Arrhythmogenic right ventricular cardiomyopathy/dysplasia (ARVC/D), characterized by non-ischemic ventricular arrhythmias originating from the right ventricle, is a significant cause of sudden death, particularly among young individuals and athletes. The pathology of ARVC/D involves progressive dystrophy of the right ventricular myocardium with fibro-fatty replacement. Current imaging techniques, however, have limited sensitivity in accurately assessing the extent and characteristics of fibro-fatty tissue in the right ventricle.

Recent advancements in digital signal processing have opened up new possibilities for echocardiographic examination, allowing for the evaluation of myocardial structure based on the acoustic properties of sound-reflecting tissues. Nonetheless, there is a need for further development in this area to optimize the assessment of myocardial ultrastructure and composition using digital signal processing techniques.

Therefore, there is a need for noninvasive systems and methods that can accurately detect and quantify the acoustic properties of myocardial tissue, thereby providing valuable information for diagnosis and risk assessment in cardiovascular diseases.

### SUMMARY OF THE DISCLOSURE

By way of introduction, the preferred embodiments described below include an easy-to-use ultrasound imaging and backscatter system is disclosed. The ultrasound imaging and backscatter system comprises an ultrasonic catheter having a longitudinal axis, a proximal end, and a distal end. Further, an ultrasonic transducer array is disposed of within the distal end of the ultrasonic catheter. The ultrasonic transducer array comprises a plurality of transducer array elements arranged on a substrate. It can be noted that the plurality of transducer array elements corresponds to a micro-electromechanical (MEMS) based Piezoelectric Micromachined Ultrasonic Transducer (pMUT). Further, the ultrasound imaging and backscatter system comprises a catheter shaft connected at one end to a handle assembly and at other end to the ultrasonic transducer array. The catheter shaft encloses an electronic flex cable which is in communication with at least one signal trace and is configured to: direct each of the plurality of transducer array elements, via the at least one signal trace, to transmit and receive, with respect to heart, ultrasound beams having a bandwidth including a predetermined fundamental mode vibration of each of the plurality of transducer array elements, such that a single array element can transmit and receive multiple fundamental mode vibrations simultaneously; receive at least one signal from the plurality of transducer array elements based on transmitting and receiving at least one ultrasound beam of the ultrasound beams, and construct at least one image of at least a portion of the heart based on the at least one signal.

Further, the ultrasound imaging and backscatter system comprises an imaging device coupled to the ultrasonic catheter using a custom dongle. The custom dongle is coupled to the handle assembly using an interposer and a flat circuit board. The custom dongle is configured to communicate ultrasound transmit pulses and ultrasound receive waveforms between the ultrasonic catheter and the imaging device. Further, the ultrasound imaging and backscatter system comprises a steering control unit positioned within the handle assembly for articulating a distal tip of the ultrasonic catheter and aligning the face of the ultrasonic transducer array towards internal views including an anterior position or a posterior position of the heart. It can be noted that the distal tip of the ultrasonic catheter is coated with a material to provide electrical isolation and transmission of ultrasound signals.

In one embodiment, a method of characterizing one or more tissue components of a scanned object in a patient, is disclosed. The method comprises steps as: positioning a catheter having a Piezoelectric Micromachined Ultrasonic Transducer Intracardiac echocardiographic (pMUT ICE) catheter adjacent to a region of interest of the scanned object. Further, the method includes receiving reflected signals from the pMUT ICE catheter; scanning the region of interest; determining one or more signal properties of the region of interest from the reflected signals. Successively, associating the one or more signal properties to predetermined signal properties of tissue components of an object similar to the scanned object. The predetermined signal properties comprise classification conditions stored in a data structure. Further, the method includes identifying one or more tissue components. The one or more tissue components includes tissue scaring, lesion assessment and measurement of tissue thickness. In one embodiment, the tissue thickness corresponds to the left ventricle, right ventricle, left atrium or right atrium thickness

In one embodiment, the reflected signals include a plurality of backscattered scan lines and the determining step includes determining signal properties for a plurality of segments from the plurality of scan lines.

In an embodiment, the method also includes measuring wall structure used in conjunction with an ICE imaging ablation and mapping catheters.

In one embodiment, a system is disclosed. The system comprises a catheter including a Piezoelectric Micromachined Ultrasonic Transducer Intracardiac echocardiographic (pMUT ICE) catheter configured to scan a region of interest. Further, the system comprises a computing device in communication with the catheter. The computing device is configured to: receive reflected signals from the pMUT ICE catheter; determine one or more signal properties of the region of interest from the reflected signals; associate the one or more signal properties to predetermined signal properties of tissue components of an object similar to the scanned object, wherein the pre-determined signal properties comprise classification conditions stored in a data structure; and identify one or more tissue components. The one or more tissue components includes tissue scaring, lesion assessment and measurement of tissue thickness. In one embodiment, the tissue thickness corresponds to the left ventricle, right ventricle, left atrium or right atrium thickness.

In one embodiment, an ultrasonic catheter is disclosed. The ultrasonic catheter comprises a body having a longitudinal axis and a distal end. Further, an ultrasonic transducer array is disposed within the distal end of the body. The ultrasonic transducer array comprises a plurality of transducer array elements arranged on a substrate. It can be noted that the plurality of transducer array elements corresponds to a micro-electromechanical (MEMS) based Piezoelectric Micromachined Ultrasonic Transducer (pMUT). Further, each of the plurality of transducer array elements comprises individual elements of multiple diameters. Further, the ultrasonic transducer array is connected in series between at least one signal trace and a common ground. Further, each transducer array element comprises a plurality of transducers, with a first group of two or more transducers in a first transducer array element and a second group of two or more transducers in the first transducer array element. Further, each of the plurality of transducer array elements are connected in parallel. Further, at least one first electrode is connected between the at least one piezoelectric layer and a signal conductor, and at least one second electrode is connected between the at least one piezoelectric layer and a ground conductor.

In one embodiment, an intracardiac echocardiographic (ICE) imaging and backscatter system is disclosed. The ICE imaging system comprises an ultrasonic catheter having a longitudinal axis, a proximal end, and a distal end. Further, a micro-electromechanical system (MEMS) based Piezoelectric Micromachined Ultrasonic Transducer (pMUT) array is disposed of within the distal end of the ultrasonic catheter. The MEMS based pMUT array comprises a plurality of MEMS based pMUT array elements arranged on a substrate. Further, the ultrasound imaging and backscatter system comprises an electronic flex cable connected at one end to a handle assembly and at other end to the MEMS based pMUT array. The electronic flex cable is in communication with at least one signal trace and is configured to: direct each of the plurality of MEMS based pMUT array elements, via the at least one signal trace, to transmit and receive, with respect to heart, ultrasound beams; receive at least one signal from the plurality of MEMS based pMUT array elements based on transmitting and receiving at least one ultrasound beam of the ultrasound beams, and construct at least one image of at least a portion of the heart based on the at least one signal.

In one embodiment, the MEMS based pMUT array comprises pMUT cells of multiple diameters to achieve a bandwidth of greater than 55%.

In one alternate embodiment, a steerable ICE catheter is disclosed with 64 or 96 parallel drive elements. The ICE catheter utilize phased array technology and are manufactured in 8 or 10 French (Fr) sizes, operating within a frequency range of 5-10 MHz. The ICE catheter enable pulsed wave and continuous wave Doppler imaging, as well as colour flow imaging. These imaging capabilities are particularly useful for evaluating pulmonary vein (PV) flows during atrial fibrillation (AF) ablation procedures. The ICE catheter's “home view” represents its neutral position in the right atrium (RA), and by executing a series of manoeuvres, comprehensive imaging of the right and left heart can be easily achieved.

In one alternate embodiment, the present invention relates to advancements in ablation catheters, specifically in the areas of high-power, short-duration (HPSD) RF delivery, single-shot RF balloons, cryoablation, and electroporation. Lesion formation with modern RF ablation catheters involves two simultaneous phases: resistive heating and conductive heating. Resistive heating occurs when the catheter tip directly contacts the tissue, resulting in superficial lesions. On the other hand, conductive heating is time-dependent and penetrates deeper into the tissue, creating transmural lesions.

In one embodiment, a cryoablation method as an alternative to RF ablation, utilizing heat withdrawal for ablation purposes, is disclosed. The cryoablation is the pioneering balloon platform designed for pulmonary vein isolation (PVI). The cryoablation method employs a single-shot delivery technique to achieve efficient ablation. Conventional cryotherapy involves the use of pressurized nitric oxide to extract heat from neighbouring tissues, achieving extremely low temperatures of up to −80° C. Cell death is induced through the formation of ice crystals, and further destruction occurs as these crystals expand during the melting process. It can be noted that point-by-point ablations are time-consuming and require high technical experience on the part of physicians. However, balloon-based ablation allows for quick, easy isolation of the PVs in a single shot.

In one embodiment, the present invention relates to electroporation, a non-thermal ablation technique that utilizes electric fields to generate Nano-holes in the cell membranes of specific cardiac tissue cells through exposure to a high-voltage field. When an adequate voltage is applied, electroporation causes irreversible effects, leading to cellular apoptosis and replacement fibrosis. The precise timeline of these changes, occurring over days to weeks, is not yet fully understood. Irreversible electroporation is also commonly known as pulsed field ablation.

In one embodiment, the present invention relates to mapping catheters utilized in the field, specifically basket or high-density contact mapping catheters. The high-density mapping catheter presents several potential benefits. While basket catheter mapping enables rapid simultaneous contact mapping of the chamber, providing a comprehensive mapping density, it has limitations in localized resolution. In contrast, this newer mapping technique allows the spines of the catheter to spread against the endocardial surface, facilitating accurate high-density contact mapping for precise localization and characterization of the focal atrial tachycardias (ATs) origin. Moreover, the ability to position multiple electrodes in a specific area of the endocardium proves advantageous, particularly in the left atrium (LA) and for mapping complex focal ATs.

In one embodiment, the present invention relates to catheter mapping and ablation as a superior treatment option compared to antiarrhythmic medications. The cornerstone of AF mapping and ablation is pulmonary vein isolation (PVI), which involves the electrical separation of the pulmonary veins (PV) from the left atrium. Catheter mapping and ablation serve as an alternative treatment option, surpassing the efficacy of antiarrhythmic medications. In AF ablation procedures, intracardiac echocardiography (ICE) plays a crucial role in performing transseptal puncture, mapping the junction between the left atrium and pulmonary veins, monitoring catheter placement, and identifying potential procedural complications. Establishing a strong endothelial contact between the ablation catheter and the endocardial surface is a vital step in ensuring the successful delivery of effective ablation lesions.

Other features and aspects of this disclosure will be apparent from the following description and the accompanying drawings.

### BRIEF DESCRIPTION OF THE DRAWINGS

The accompanying drawings illustrate various embodiments of systems, methods, and embodiments of various aspects of the disclosure. Any person of ordinary skill in the art will appreciate that the illustrated element boundaries (e.g., boxes, groups of boxes, or other shapes) in the figures represent one example of the various boundaries representative of the disclosed invention. It may be that in some examples one element may be designed as multiple elements or that multiple elements may be designed as one element. In other examples, an element shown as an internal component of one element may be implemented as an external component in another and vice versa. Furthermore, elements may not be drawn to scale. Non-limiting and non-exhaustive descriptions of the present disclosure are described with reference to the following drawings. The components in the figures are not necessarily to scale, emphasis instead being placed upon the illustrated principles.

Various embodiments will hereinafter be described in accordance with the appended drawings, which are provided to illustrate and not to limit the scope of the disclosure in any manner, wherein similar designations denote similar elements, and in which:

FIGS. 1 and 2 illustrate a prior art imaging system, for acquiring two-dimensional image information;

FIG. 3 illustrates a schematic diagram of an ultrasonic imaging system, according to an embodiment of the present disclosure;

FIG. 4 illustrates a schematic diagram of a front view of an ultrasonic catheter and a custom dongle, according to an embodiment of the present disclosure;

FIG. 5 illustrates a schematic view of the ultrasonic catheter with a distal tip being diverted to an anterior position and a posterior position using a steering control unit, according to an embodiment of the present disclosure;

FIG. 6 illustrates a multi-channel electronic communication between the imaging device and an ultrasonic transducer array of the ultrasonic catheter, according to an embodiment of the present disclosure;

FIG. 7 illustrates a sectional view of a distal end of the ultrasonic catheter with a plurality of transducer array elements, according to an embodiment of the present disclosure;

FIGS. 8A-8B illustrate schematic diagrams of the human heart with the ventricle and septum walls illustrated according to an embodiment of the present disclosure;

FIG. 9 illustrates a block diagram of the signal analyser logic, according to an embodiment of the present disclosure;

FIG. 10 illustrates a schematic diagram showing the signal flow of the signal analyser logic, according to an embodiment of the present disclosure;

FIG. 11 illustrates a graph showing a received RF signal, according to an embodiment of the present disclosure;

FIG. 12 illustrates a sectional view of a heart ventricle wall, according to an embodiment of the present disclosure.

### DETAILED DESCRIPTION

The components of the embodiments as generally described and illustrated in the figures herein can be arranged and designed in a wide variety of different configurations. Thus, the following more detailed description of various embodiments, as represented in the figures, is not intended to limit the scope of the present disclosure, but is merely representative of various embodiments. While various aspects of the embodiments are presented in drawings, the drawings are not necessarily drawn to scale unless specifically indicated.

Some embodiments of this disclosure, illustrating all its features, will now be discussed in detail. The words “comprising,” “having,” “containing,” and “including,” and other forms thereof, are intended to be equivalent in meaning and be open-ended in that an item or items following any one of these words is not meant to be an exhaustive listing of such item or items, or meant to be limited to only the listed item or items.

It must also be noted that as used herein and in the appended claims, the singular forms “a,” “an,” and “the” include plural references unless the context dictates otherwise. Although any systems and methods similar or equivalent to those described herein can be used in the practice or testing of embodiments of the present disclosure, the preferred systems, and methods are now described. The terms “proximal” and “distal” are opposite directional terms. For example, the distal end of a device or component is the end of the component that is furthest from the practitioner during ordinary use. The proximal end refers to the opposite end, or the end nearest the practitioner during ordinary use.

Embodiments of the present disclosure will be described more fully hereinafter with reference to the accompanying drawings in which like numerals represent like elements throughout the several figures, and in which example embodiments are shown. Embodiments of the present disclosure may, however, be embodied in alternative forms and should not be construed as being limited to the embodiments set forth herein. The examples set forth herein are non-limiting examples and are merely examples among other possible examples.

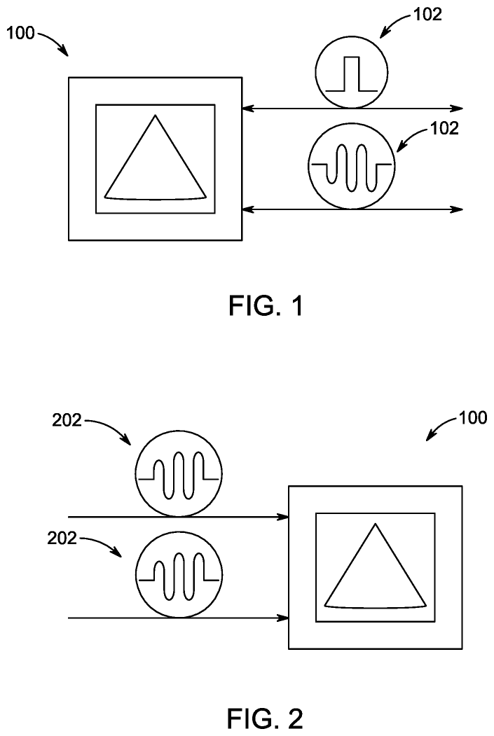

FIGS. 1 and 2 illustrate a prior art imaging system 100. The imaging system 100 may be used for diagnosis and/or treatment in combination with another imaging modality, such as an x-ray, fluoroscopy, magnetic resonance, computed tomography, or optical system. It can be noted that imaging modalities scan a patient for generating images to assist a physician. Further, the imaging system 100 provides an ultrasound transmit pulse 102 and an ultrasound receive path 202, for connection to an ultrasonic transducer (not shown). The ultrasound transmit pulse 102 may transmit ultrasound signals from the imaging system 100 towards an object such as heart of a patient. Further, the ultrasound receive path 202 may create a waveform based at least on the ultrasound signals. Thereafter, the imaging system 100 may convert the received ultrasound signals or ultrasound information to a two-dimensional (2D) image of the object or a portion of the object.

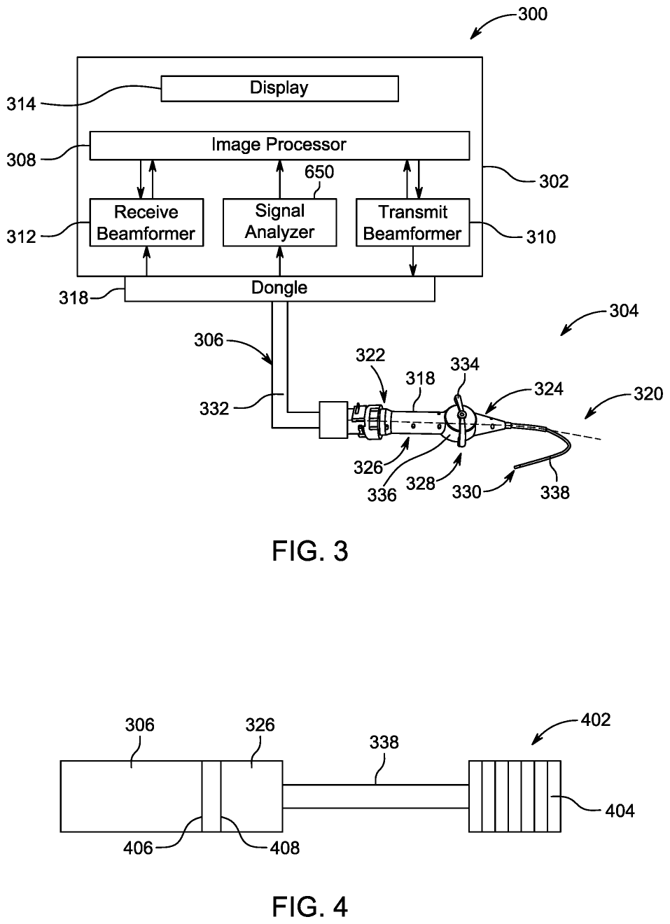

FIG. 3 illustrates a schematic diagram of an ultrasound imaging and backscatter system 300, according to an embodiment of the present disclosure. FIG. 3 is described in conjunction with FIGS. 4-12B.

The ultrasound imaging and backscatter system 300 may be performed for electrophysiology (EP). The ultrasound imaging and backscatter system 300 may be used for diagnosis and/or treatment in combination with another imaging modality, such as an x-ray, fluoroscopy, magnetic resonance, computed tomography, or optical system. Both imaging modalities may scan a patient for generating images to assist a physician. The data from the different modalities may be aligned by locating the markers with a known spatial relationship to the ultrasound scan in the images of the other modality. In other embodiments, the ultrasound imaging and backscatter system 300 may use a catheter without the markers and/or without another imaging modality. In one embodiment, the ultrasound imaging and backscatter system 300 may utilize a microelectromechanical (MEMS) transducer array defined as piezoelectric micro-machined ultrasound transducer (pMUT) or other types of MEMS transducers, interconnected using matched flexible circuits. In one embodiment, the ultrasound imaging and backscatter system 300 may correspond to an intracardiac echocardiographic (ICE) imaging system. In one embodiment, the ultrasound imaging and backscatter system 300 may correspond to an endovascular MEMS ultrasonic transducer utilizing a high-density flexible circuit for all transmission and electrical interconnects. In one embodiment the ultrasound imaging and backscatter system 300 analyses the spectral backscattered radio frequency (RF) data. In one embodiment, the ultrasound imaging and backscatter system 300 may be employed to treat patient with cystic fibrosis (CF). It can be noted that the use of the high-density flexible circuits may enable highly repeatable and stable transmission and return signals. Further, the high density flexible circuit transmission lines may transmit electrical energy from one end to another distal end of the ultrasound imaging and backscatter system 300.

Referring to FIG. 3, the ultrasound imaging and backscatter system 300 may comprise an imaging device 302 coupled to an ultrasonic catheter 304 via a communication channel 306. In one embodiment, the communication channel 306 may be a custom dongle with a cable and bus connections or multiple connections. Hereinafter, the communication channel 306 may be referred to as the custom dongle 306.

The ultrasonic catheter 304 may be disposed within a chamber of a heart of a patient and the imaging device 302 may receive at least one signal from the ultrasonic catheter 304. The at least one signal may be communicated from the ultrasonic catheter 304 to the imaging device 302 via the custom dongle 306. Further, the imaging device 302 may comprise an image processor 308, a transmit beamformer 310, a receive beamformer 312, a signal analyzer 316 and a display 314.

The image processor 308 may be configured to generate a two-dimensional (2D) image according to data received from the ultrasonic catheter 304. In one embodiment, the image processor 308 may be configured to receive a focused signal from the receive beamformer 312. The image processor 308 may render the data to construct an image or sequence of images. In one embodiment, the image may be three dimensional (3D) representation, such as a two-dimensional image rendered from a user or a processor selected viewing direction. In one embodiment, the image processor 308 may be a detector, filter, processor, application-specific integrated circuit, field-programmable gate array, digital signal processor, control processor, scan converter, three-dimensional image processor, graphics processing unit, analog circuit, digital circuit, or combinations thereof. The image processor 308 may receive beamformed data and may generate images, to display on the display 314. It can be noted that the generated images are associated with a two-dimensional (2D) scan. Alternatively, the generated images may be three-dimensional (3D) representations.

The image processor 308 may be programmed for hardware accelerated two-dimensional re-constructions. The image processor 308 may store processed data of the at least one signal and a sequence of images in a memory. In one embodiment, the memory may be a non-transitory computer-readable storage media. The instructions for implementing the processes, methods and/or techniques discussed herein are provided on the computer-readable storage media or memories, such as a cache, buffer, RAM, removable media, hard drive, or other computer-readable storage media. Non-transitory computer-readable storage media include various types of volatile and non-volatile storage media. The functions, acts, or tasks illustrated in the figures or described herein are executed in response to one or more sets of instructions stored in or on a computer readable storage media. The functions, acts, or tasks are independent of the particular type of instructions set, storage media, processor, or processing strategy and may be performed by software, hardware, integrated circuits, firmware, micro code, and the like, operating alone or in combination.

The transmit beamformer 310 may be configured for transmission of the electrical signal or electrical impulse in a form of at least one signal towards the ultrasonic catheter 304. The receive beamformer 312 may be configured to receive an electrical signal or electrical impulse from the ultrasonic catheter 304. In one embodiment, the transmit beamformer 310 and the receive beamformer 312 may facilitate transmit beamforming technique to focus energy towards a receiver to improve a signal to noise (SNR) of the at least one signal and then transmit the at least one signal to the image processor 308.

The display 314 may be configured to screen the image or sequence of images during or after the data is rendered, by the image processor 308. The image may be three dimensional (3D) representation, such as a two-dimensional image rendered from a user or a processor selected viewing direction. Alternatively, the image may be one or more two-dimensional images representing planes in the volume. In one embodiment, the display 314 may be a part of imaging device 302 or may be remote, such as a networked display. In one embodiment, the display 314 may be a cathode ray tube (CRT), liquid crystal display (LCD), a projector, a plasma, or other now known or later developed display device.

The ultrasonic catheter 304 may be in electronic communication with the imaging device 302 for transmission and receiving of ultrasound signals to and from an arterial wall of a vascular system. In one embodiment, the ultrasonic catheter 304 may be configured to visualize standard echocardiography views of the heart, such as in a standard version, a right atrium may be visualized. The ultrasonic catheter 304 may be employed in transseptal catheterization for several percutaneous interventions, including left heart catheter ablation, atrial septal defect closure for effective alternative to surgical intervention. Further, the ultrasonic catheter 304 may comprise a body 318 having a longitudinal axis 320, a proximal end 322, a distal end 324, a handle assembly 326, a steering control unit 328, a distal tip 330, and a dongle cable 332.

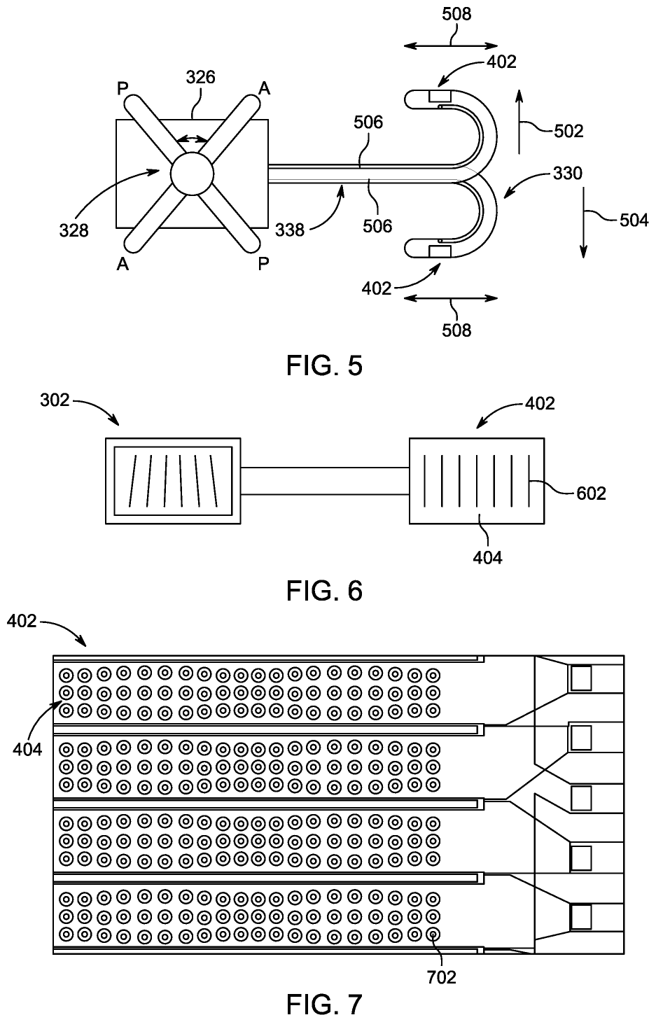

The handle assembly 326 may be positioned between the proximal end 322 and the distal tip 330 of the ultrasonic catheter 304. Further, the steering control unit 328 may be positioned within the handle assembly 326. The steering control unit 328 may be provided for articulating the distal tip 330 of the ultrasonic catheter 304. Further, the steering control unit 328 may align face of an ultrasonic transducer array (not shown) towards different positions with respect to the ultrasonic catheter 304. Further, the steering control unit 328 may comprise a steering handle 334 and a housing 336 enclosing an actuator (not shown) and a steering hub (not shown). It can be noted that an internal friction occurs between the actuator and the steering hub, and between the actuator and the housing 336, which causes the ultrasonic catheter 304 to retain its adjusted configuration without operator attention. The steering handle 334 may be rotated to facilitate positioning of the distal tip 330 of the ultrasonic catheter 304. The movement of the distal tip 330 by the steering control unit 328 is shown in FIG. 5. In one embodiment, the steering handle 334 may be rotated to position the distal tip 330 inside the chamber of the heart of the patient. In one embodiment, the steering control unit 328 may comprise a set of steering lines controlled by a steering actuator to articulate bidirectional a distal segment of the ultrasonic catheter 304. It can be noted that the steering handle 334 may be rotated from 0 degrees to +/−45 degrees. A catheter shaft 338 may be coupled to the handle assembly 326 at one end and to the distal tip 330 of the ultrasonic catheter 304 at the other end. Further, the catheter shaft 338 may enclose an electronic flex cable (not show) and a plurality of steering cables (not shown). In one embodiment, the electronic flex cable may be a stainless steel cable. The electronic flex cable may be coupled at one end to the handle assembly 326 and at other end to the ultrasonic transducer array. The electronic flex cable and the plurality of steering cables may be described later in conjunction with FIGS. 6A-7. Further, the ultrasonic catheter 304 is described in conjunction with FIG. 4.

Referring to FIG. 4, the ultrasonic catheter 304 may comprise an ultrasonic transducer array 402, a substrate 404, an interposer 406, and a flat circuit board 408. The ultrasonic transducer array 402 may be disposed within the distal tip 330 of the ultrasonic catheter 304. In one embodiment, the ultrasonic transducer array 402, the substrate 404, the interposer 406, and the flat circuit board 408 may correspond to flexible printed electronic circuits for communicating from the distal end 324 of the ultrasonic catheter to the dongle cable 332 or directly to the ultrasound imaging and backscatter system 300. The ultrasonic transducer array 402 may be disposed over the substrate 404 towards the distal end 328 of the ultrasonic catheter 304. It can be noted that the ultrasonic transducer array 402 may correspond to MEMS based pMUT array. The handle assembly 326 may be coupled to the proximal end 322 of the ultrasonic catheter 304, using the interposer 406 and the flat circuit board 408. In one embodiment, the handle assembly 326 may be coupled to the dongle cable 332 using a catheter handle (not shown) and the interposer 406. The catheter handle will be described later in conjunction with FIG. 6B to FIG. 6D. It can be noted that the interposer 406 may be coupled to the custom dongle 306 and the flat circuit board 408 may be coupled to the proximal end 322 of the ultrasonic catheter 304, towards the handle assembly 326.

As shown in FIGS. 3-4, the catheter shaft 338 may be coupled between the handle assembly 326 and the ultrasonic transducer array 402. The electronic flex cable inside the catheter shaft 338 may receive the at least one signal from the ultrasonic transducer array 402 and the received signal may be communicated back to imaging device 302 via the custom dongle 306. The electronic flex cable may be coupled at one end to the handle assembly 326 and at other end to the ultrasonic transducer array 402. It can be noted that the ultrasonic transducer array 402 may receive electrical signals from the imaging device 302 via the custom dongle 306 and the electronic flex cable. It can also be noted that the ultrasonic transducer array 402 may transmit the at least one signal back to the imaging device 302 to further analyze the at least one signal for image generation. Further, the ultrasonic catheter 304 may be coupled to the ultrasonic imaging device 302 using the dongle cable 332.

Referring to FIG. 5, a schematic view of the ultrasonic catheter 304 with the distal tip 330 being diverted to an anterior position 502 and a posterior position 504 using the steering control unit 328 is disclosed, according to an embodiment of the present disclosure.

The steering control unit 328 may be positioned within the handle assembly 326 for articulating the distal tip 330 of the ultrasonic catheter 304 and for aligning the face of the ultrasonic transducer array 402 towards internal view including, the anterior position 502 or the posterior position 504 of the heart. It can be noted that the distal tip 330 of the ultrasonic catheter 304 may correspond to a tip of the catheter shaft 338 of the ultrasonic catheter 304. Further, the ultrasonic transducer array 402 may be disposed of within the distal tip 330 of the ultrasonic catheter 304. It can be noted that a cable connecting the distal end 324 of the catheter handle to the distal tip 330 may be the catheter shaft 338. In one embodiment, the ultrasonic transducer array 402 may be positioned towards the internal views including anterior position 502 and the posterior position 504 of the heart. The distal tip 330 of the ultrasonic catheter 304 may be curved towards the distal end 324. In one embodiment, the distal tip 330 of the ultrasonic catheter 304 may be coated with a material to provide electrical isolation and transmission of ultrasound signals. Further, the catheter shaft 338 in communication between the distal tip 330 and the distal end 324 of the ultrasonic catheter 304, may transmit electrical signals or pulses to the distal tip 330 of the ultrasonic catheter 304, and the ultrasonic transducer array 402 may transmit back acoustic echo to the imaging device 302 via the catheter shaft 338 and the custom dongle 306.

Further, the ultrasonic catheter 304 may comprise a plurality of steering cables 506 that may be diverted to the anterior position 502 and the posterior position 504 using the steering control unit 328, as shown in FIG. 5. The plurality of steering cables 506 may be housed within the catheter shaft 338. In one embodiment, at least two steering cables of the plurality of steering cables 506 may be diverted towards at least two distal tips with ultrasonic transducer arrays, using the steering control unit 328. Further, the catheter shaft 338 having a gradient durometer 508 of Pebax material towards the distal tip 330 of the ultrasonic catheter 304. It can be noted that the gradient durometer 508 of Pebax material may be hard and rigid towards the proximal end 322 and softer towards the distal end 324 of the catheter shaft 338. In one embodiment, the catheter shaft 338 may be softer for at least 6 to 8 inches towards the distal end 324. It can be noted that the distal tip 330 may have the softer Pebax material. Further, the plurality of steering cables 506 may be configured to bend or tilt the distal tip 330, when the steering handle of the steering control unit 328 is rotated clockwise or counter clockwise. It can be noted that the actuator of the steering control unit 328 may pull a steering cable of the plurality of steering cables 506, when inserted inside the heart. The steering cable of the plurality of steering cables 506 may then bend the distal tip 330 towards the anterior position and the posterior position inside the heart. In one embodiment, at least two steering cables of the plurality of steering cables 506 may be bent using the steering control unit 328.

In one embodiment, the plurality of steering cables 506 can be made of synthetic materials, such as nylon or similar synthetic fibres, or plastics material, such as urethane, Teflon®, Kynar®, Kevlar®, polyethylene, multi-stranded nylon, or gel-spun polyethylene fibres. For example, the plurality of steering cables 506 may be a multi-stranded Spectra® brand nylon line sold as Spiderwire® fishing line (10 lbs. test).

Referring to FIG. 6 a multi-channel electronic communication between the ultrasonic imaging device 302 and the ultrasonic PMUT transducer array 402 is disclosed, according to an embodiment of the present disclosure. The ultrasonic transducer array 402 may comprise a plurality of transducer array elements 602 arranged on the substrate 404. Further, each of the plurality of transducer array elements 602 may provide a wide bandwidth of an individual focused beam. The ultrasonic transducer array 402 may be coupled to the ultrasonic imaging device 302 using the dongle cable 332, as described earlier. The MEMS based ultrasonic transducer array 402 disposed within the distal end 324 of the ultrasonic catheter 304 may transmit the at least one signal via the electronic flex cable 602 inside the catheter shaft 338 to the imaging device 302. The at least one signal may be the acoustic echo transmitted from the ultrasonic transducer array 402. It can be noted that the acoustic echo of acoustic energy may be received from a face of the ultrasonic transducer array 402 and received at the image processor 308.

Further, the plurality of steering cables 506 may be configured to direct each of the plurality of transducer array elements 602, via the at least one signal trace, to transmit and receive, ultrasound beams. The ultrasound beams may have a bandwidth including a predetermined fundamental mode vibration of each of the plurality of transducer array elements 602, such that a single array element can transmit and receive multiple fundamental mode vibrations simultaneously. It can be noted that the plurality of transducer array elements 602 may transmit and receive the ultrasound beams with respect to the heart or at least a portion of the heart. Further, the electronic flex cable 602 inside the catheter shaft 338 may be configured to receive at least one signal from the plurality of transducer array elements 602 based on transmitting and receiving at least one ultrasound beam of the ultrasound beams. The imaging device 302 may be further configured to construct at least one image of at least the portion of the heart based on the at least one signal. It can be noted that the electronic flex cable may be configured to the transmit beamformer 310 and receive beamformer 312 to display a two-dimensional (2D) image information of the heart or the at least portion of the heart.

In one embodiment, the plurality of transducer array elements 602 may correspond to a micro-electromechanical (MEMS) based Piezoelectric Micromachined Ultrasonic Transducers (pMUTs). The catheter shaft 338 may be connected to the handle assembly 326 at one end and to the ultrasonic transducer array 402 at other end. The electronic flex cable 602 inside the catheter shaft 338 may be in communication with the at least one signal trace. It can be noted that the electronic flex cable 602 may be further communicate to the transmit beamformer 310 and the receive beamformer 312, via the custom dongle 306 to display a two-dimensional (2D) image information of the heart to be scanned.

Referring to FIG. 7, a sectional view of the distal end 324 of the ultrasonic catheter 304 having the ultrasonic pMUT transducer array 402 with the plurality of transducer array elements 602, is disclosed, according to an embodiment of the present disclosure.

The distal end 324 of the ultrasonic catheter 304 may be provided with the ultrasonic transducer array 402 having the plurality of transducer array elements 602. Further, each of the plurality of transducer array elements 602 may have a plurality of individual transducer 702 arranged in a manner to provide a wide bandwidth of the individual focused beam. In one embodiment, the ultrasonic transducer array 402 may be constructed from a pMUT array containing individual elements of different diameters. In one embodiment, to achieve wider bandwidth with pMUT arrays, multiple diameters of pMUT cells may be integrated into one element. It can be noted that by arranging pre-shaped pMUTs with different diameters, a broader bandwidth can be realized through the complex interaction between the individual pMUT elements. In one embodiment, the pMUT cells of multiple diameters may achieve a bandwidth of greater than 55%. For example, in 3 elements, there are 5 different dome diameters, and each array is of a different size, such as 300 μm.

Further, the ultrasonic transducer array 402 may correspond to pMUT and the plurality of transducer array elements 602 may correspond to a plurality of pMUT elements. In one embodiment, the plurality of pMUT elements may be directed to transmit and receive, the ultrasound beams having the bandwidth including the predetermined fundamental mode vibration of each of the plurality of pMUT elements, such that a single pMUT element can transmit and receive multiple fundamental mode vibrations simultaneously. Further, the electronic flex cable 602 inside the catheter shaft 338 receives the at least one signal from the plurality of pMUT elements. It can be noted that the at least one signal may correspond to the at least one ultrasound beam. The at least one signal may be transmitted to the ultrasonic imaging device 302 for further processing in the image processor 308. The image processor 308 may construct the at least one image of the heart. It can be noted that the plurality of pMUT elements may be used to create the individual focused beam.

In one alternate embodiment, the ultrasonic transducer array 402 may include a cover portion that presents a circular cross-section. It can be noted that a feature of ultrasonic transducer array 402 is typical in ultrasonic imaging catheters. Due to the severe space restrictions imposed by the small diameter of intracardiac catheters, the ultrasonic transducer array is typically limited to a linear phased array made up of several individual transducer elements, such as 64 transducers or elements. The transducers have a flat surface from which sound is omitted and echoed sound is received. As is well known in the art, the individual transducer elements are pulsed by an ultrasound control system so that the emitted sound waves are constructively combined into a primary beam. By varying the time at which each transducer element is pulsed, the ultrasound imaging and backscatter system 300 may render the individual beams into a focused image which can be swept through an arc in order to obtain the 2D image. As a result, the ultrasonic transducer array 402 emits ultrasound along a plane that is perpendicular to the face of the transducer arrays. Thus, the ultrasonic transducer array 402 emits sound along a plane that is perpendicular to the assembly.

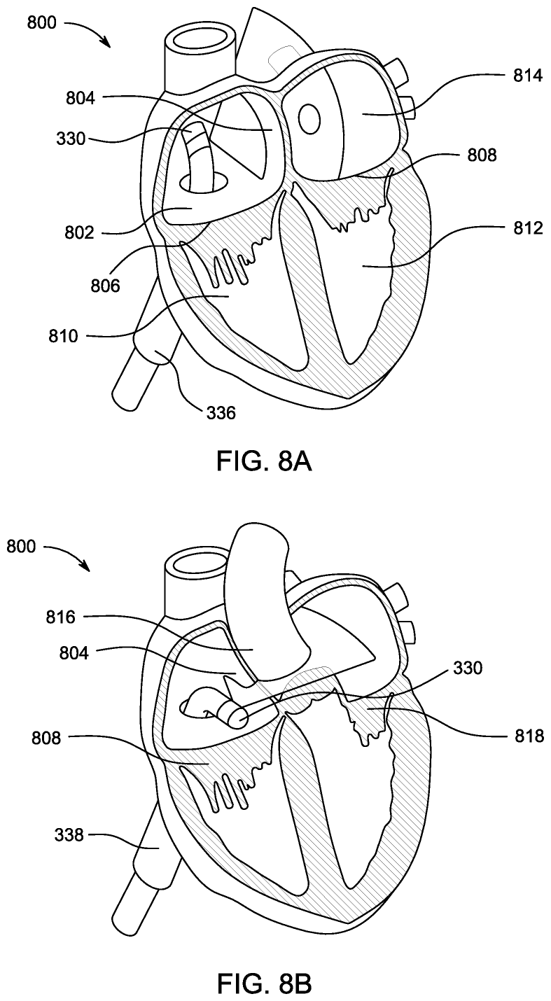

Referring to FIGS. 8A-8B, cross sectional images of a heart 800 with the ultrasonic catheter 304 positioned within a right atrium 802 of the heart 800, are disclosed. The distal tip 330 of the ultrasonic catheter 304 may be inserted into the right atrium 802 via an inferior vena cava (not shown). In order to perform an adequate imaging of an interatrial septum (IAS) 804 and its neighbouring structures, two standardized views may be used. The movement of the distal tip 330 of the ultrasonic catheter 304 within the right atrium 802 may be controlled by the steering control unit 328. The steering handle 334 of the steering control unit 328 may be rotated to facilitate positioning of the distal tip 330 of the ultrasonic catheter 304 within the heart 800 of the patient. In one embodiment, the steering handle 334 may be steered clockwise to position the distal tip 330 of the ultrasonic catheter 304 to a rear view of the right atrium 802. In another embodiment, the steering handle 334 may be steered counter clockwise to position the distal tip 330 of the ultrasonic catheter 304 to a front view of the right atrium 802. Further, the movement of the steering handle 334 clockwise or counter clockwise, may allow an imaging window to move from a posterior view to an anterior view or vice versa.

Further, a flexible sheath may be introduced into the patient's vascular structure via a femoral vein (not shown) to properly place the ultrasonic transducer array 402 into position for imaging the right atrium 802 and a bicuspid valve 806. Using a fluoroscopic imaging to monitor the ultrasonic catheter 304 position, a clinician may advance the distal end 324 of the ultrasonic catheter 304 into the right atrium 802. In order to guide the ultrasonic catheter 304 through turns in the patient's vascular structure, the clinician may rotate the steering handle 334 clockwise or counter clockwise to allow the imaging window to move towards the internal views including the anterior position 502 to the posterior position 504 and vice versa. Once the distal tip 330 of the ultrasonic catheter 304 is in the right atrium 802, the clinician may rotate the steering handle 334 so as to introduce an acute bend in the flexible sheath to direct the ultrasonic transducer array 402 through a tricuspid valve 808 and into a right ventricle 810, as shown in FIG. 8A. In this position, a field of view of the ultrasonic transducer array 402 may include portions of the right ventricle 810, the IAS 804, a left ventricle 812, a left atrium 814, the bicuspid valve 806, and a left ventricular wall. In one embodiment, when the ultrasonic transducer array 402 is directed by rotating the steering handle 180 degrees clockwise, the right ventricle 810 and right ventricular wall may be imaged. It can be noted that the clinician may twist the ultrasonic catheter 304, while positioned within the heart 800 as illustrated in FIGS. 8A-8B, the transducer array 402 may swing about an axis, which could injure the tricuspid valve 808 or cause the ultrasonic transducer array 402 to strike the IAS 804. FIG. 8B shows a perpendicular short-axis view to visualize interior parts of the IAS 804. The interior parts of the IAS 804 include an aorta 816, the IAS 804, a mitral valve 818 towards the left ventricle 812.

In one exemplary embodiment, the standard view is obtained by placing the ultrasonic catheter 304 in a mid-right atrium 802 and the ultrasonic transducer array 402 in a neutral position facing a tricuspid valve 808. The standard view provides imaging of the right atrium 802, the tricuspid valve 808, right ventricle 810, and typically an oblique or short-axis view of the aortic valve.

Further, when the ultrasonic catheter 304 may be rotated clockwise the aortic valve in long axis and the right ventricle 810 outflow tract is viewed. In this view, the tricuspid valve that is closer to the ultrasonic transducer array 402 or the distal tip 330 is a non-coronary cusp, which is in close relationship to a membranous septum and a para-hisian region, whereas the opposite is a right coronary cusp, which is the most anterior of the aortic cusps, directly posterior to the right ventricle 810 outflow tract infundibulum and pulmonic valve. The left ventricle 812 is visualized anterior to the most septal portion of the right atrium 802, and the opening of the coronary sinus becomes evident. In this view, the long axis of the left ventricle 812 outflow tract is identified, and the posterior left ventricle 812 is in view just below the non-coronary cusp.

Further, an additional clockwise rotation of the ultrasonic catheter 304 allows visualization of the mitral valve 818 and the IAS 804, with the left atrial appendage anteriorly and the coronary sinus posteriorly. The left atrium 814 appendage is examined for the presence of thrombus at its ostium, and mitral regurgitation may be assessed using a colour Doppler.

In one embodiment, most catheters used in intravascular applications, particularly those with ultrasound transducers, are at least about 10 French in diameter. The electronics and wires needed for ultrasound transducer arrays have made it impractical and expensive to reduce the size of such catheters below about 10 French. Nevertheless, there are benefits in reducing the diameter of the catheter, and technology advances may enable the electronics and control structures to be further reduced in size. The bundling arrangement of the coaxial cables, steering and pivot cables and steering and pivot mechanisms described in more detail below, make it possible to effectively reduce the diameter below about 10 French, to 4, 6, or 8 French or even 3 French (approximately 1 mm).

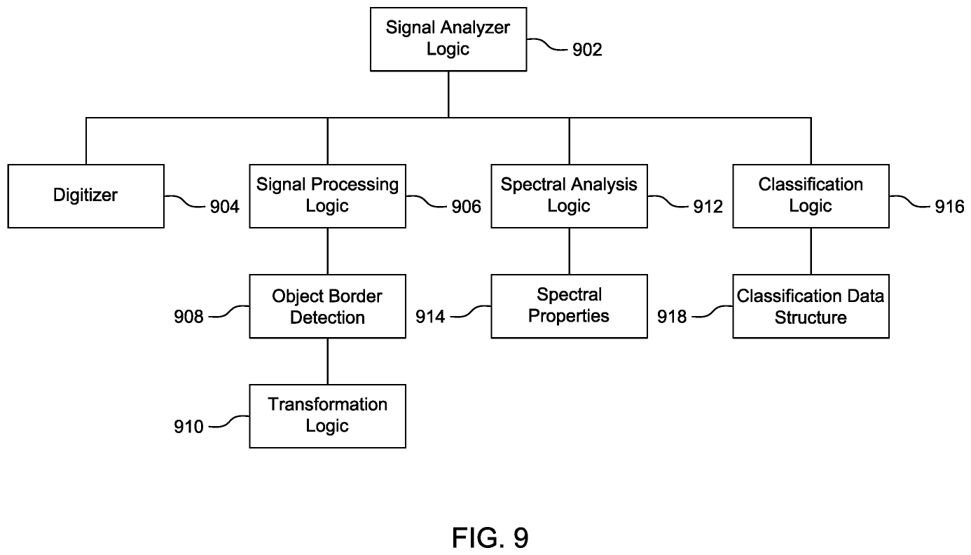

Referring to FIG. 9, a signal analyzer logic 902 for processing and analyzing radio frequency ultrasound data, is disclosed. It will be appreciated that the signal analyzer logic 902 may be embodied as part of an ultrasound imaging console, an ablation system console, or as part of a separate system that receives raw radio frequency data from the ultrasound catheter 304. If the radio frequency data is in analog form, a digitizer 904 may be provided to digitize the data. A signal processing logic 906 is configured to process each individual beam of the ultrasound data and transform it to a format that can be analyzed. To reduce processing time, an object border detection 908 may be used to determine the location of the borders of the object being scanned.

After border detection, the individual beam data is transformed. In some embodiments, the border detection may be performed after transformation. A transformation logic 910 may be configured to transform the remaining individual beam data into a suitable format for analysis. In general, the transformed format should match the same format used to build the pre-determined signal properties of the object component. In one embodiment, the transformation logic 910 may transform the data to a power spectrum plot of frequency versus power output Various transformation algorithms include a Fourier transformation, Welch periodograms, and autoregressive modeling. Other types of transformations may include transforming the data to wavelets that provide an image with frequency and time information. For example, other signal processing techniques may include wavelet decomposition or curvelet decomposition to deliver parameters that are relevant for discrimination between tissue types while not being influenced by the system transfer function of the imaging system and probe. Another trans formation includes using impedance, rather than frequency, which gives an image of acoustic impedance. In this format, different tissue components have different impedance properties that provide different signal reflections.

With further reference to FIG. 9, a spectral analysis logic 912 may analyze the power spectrum of the individual beam data to determine its spectral properties 914. As mentioned previously, spectral properties or parameters may include maximum power, frequency at the maximum power, minimum power, the frequency at the minimum power, the slope, y-intercept, mid-band fit, and integrated backscatter. The spectral parameters 914 are then inputted to a classification logic 916 that attempts to classify the spectral parameters associated to a particular individual beam segment with previously measured spectral parameters from a known tissue component. As mentioned above, the signal analyzing techniques need not be limited to spectral analysis and autoregressive coefficients, but could entail use of wavelet decomposition or curvelet decomposition to deliver parameters that may be used by the classification logic 916 to discriminate between tissue types.

In one embodiment, a classification data structure 918 may contain a statistical classification of measured or observed spectral properties (and/or other properties) associated with particular types of tissue and/or ablated tissue components. The classification data structure 918, in one embodiment, is previously generated from laboratory studies that correlate ultrasound data analysis of ablated tissue samples with their corresponding histology sections.

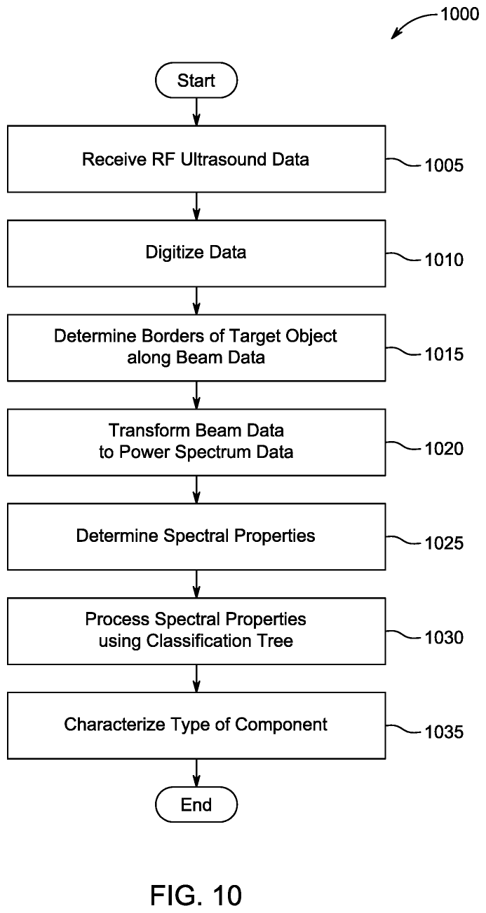

Referring to FIG. 10, a method 1000 for analyzing ultrasound signals and identifying the type of tissue component that corresponds to the signals, is disclosed. The illustrated elements denote “processing blocks and represent computer software instructions or groups of instructions that cause a computer or processor to perform an action(s) and/or to make decisions. Alternatively, the processing blocks may represent functions and/or actions performed by functionally equivalent circuits such as a digital signal processor circuit, an application specific integrated circuit (ASIC), or other logic device. The diagram does not depict syntax of any particular programming language. Rather, the diagram illustrates functional information one skilled in the art could use to fabricate circuits, generate computer software, or use a combination of hardware and software to perform the illustrated processing. It will be appreciated that electronic and software applications may involve dynamic and flexible processes such that the illustrated blocks can be performed in other sequences different than the one shown and/or blocks may be combined or separated into multiple components. They may also be implemented using various programming approaches Such as machine language, procedural, object oriented, artificial intelligence, or other techniques. This applies to all methodologies described herein.

In other embodiments, the steps of the method 1000 may be employed to analyze imaging signals received from another imaging modality and to identify the type of tissue component that corresponds to the signals. In some embodiments, instead of receiving ultrasound data and analyzing ultrasound imaging properties such as spectral properties, the system 100 may instead receive imaging data specific to the imaging data specific to the particular type of imaging modality and analyze imaging properties specifically associated with the type of imaging modality used, in light of the relevant secondary parameters.

At first, analysis may begin as RF ultrasound data may be received, at step 1005. The RF ultrasound data may be received in real time during a scan or after a scan is completed. Further, the RF ultrasound data may be digitized, at step 1010. In one embodiment, if the RF ultrasound data is still in the raw radio frequency form, it may be digitized. In one embodiment, the digitized data is analyzed along an individual beam, in one or more segments. The embodiment of FIG. 10 illustrates the analysis of one segment of data. Although not shown in FIG. 10, the processing repeats for each segment of the individual beam and repeats for other individual beams until complete or until processing is stopped. Optionally, the process may allow for changing the properties of how the individual beam is segmented Such as defining various sizes and intervals of segments.

Successively, determine borders of target object along beam data, at step 1015. For the individual beam being analyzed, a border detection algorithm may be used to identify the borders of the target object and the analysis can be focused on the individual beam data corresponding to the target object. Since the scan is not intravascular in this example, the individual beam that passes through the target object may pass through two or more walls of the object. For example, the region of interest for the scan is a vascular object, such as, a carotid body. The border detection algorithm may identify the borders of the carotid body and/or an adjacent vessel. A number of individual beams may pass through two walls of the carotid body. Thus, the border detection would attempt to search and identify at least two borders along the individual beam. Many different border detection methods are available including analyzing signal properties of the scan line, reconstructing an image from the ultrasound data and detecting borders from the image data, and other methods. Individual beam data outside the borders of the target object may be ignored or removed from analysis if desired.

The individual beam can be segmented and analyzed by segment. Further, transform beam data to power spectrum data, at step 1020. In one embodiment, the signal data from a segment is transformed to a power spectrum form. Successively, determine spectral properties, at step 1025. The spectral properties may be determined from the power spectrum which may include they intercept, maximum power, mid-band fit, minimum power, frequencies at maximum and minimum powers, slope of regression line, integrated backscatter, and/or other properties from the power spectrum. Other properties may be determined from wavelet decomposition or curvelet decomposition techniques. The spectral properties and/or other properties of the individual beam data are then compared to corresponding pre-determined properties of known tissue components to determine which type of component best matches the individual beam spectral properties. The tissue components may include tissue scaring, lesion assessment and measurement of tissue thickness.

In one embodiment, the pre-determined tissue properties are structured as a classification tree generated from statistical analysis of how the properties correlate to a type of tissue component. Successively, process spectral properties using classification tree, at step 1030. The individual beam spectral properties are then processed through the tree, traversing branches based on how the spectral properties meet the conditions of the branch nodes. The tree is traversed to a leaf node that identifies a type of tissue component. Successively, characterize type of component, at step 1035. The spectral properties of the individual beam segment are then characterized as this type of component. In one embodiment, the method 1000 may also output an assessment score of the observed ablation level within the tissue component.

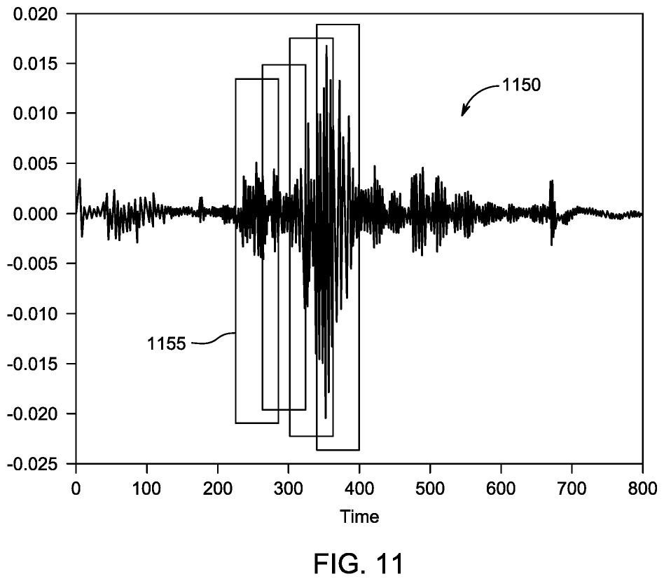

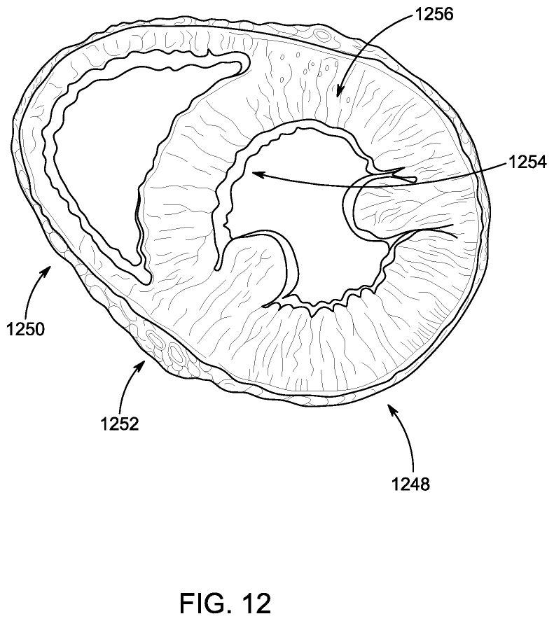

Referring to FIG. 11, a graph showing the received RF signal, is disclosed. It can be noted that data of one individual beam 1150 or referred as the RF signal is plotted as voltage over time. The individual beam 1150 can be analyzed in segments represented by multiple windows, such as window 1155. The data within the window 1155 is transformed in this embodiment to a power spectrum density plot. Signal properties of the individual beam 1150 from the window 1155 are determined from the power spectrum. Signal properties, in this case also referred to as spectral properties, may include the y-intercept, maximum power, mid-band fit, minimum power, frequencies at maximum and minimum powers, slope of regression line, integrated backscatter, or combinations of these or others.

Referring to FIG. 12, a sectional view of heart ventricle wall 1248, is disclosed. The heart ventricle wall 1248 includes a Myocardium 1250, an Epicardium 1252, an Endocardium 1254 and an area of scarring 1256 caused by myocardial fibrosis. Identification of wall cardiomyopathy may include: Transthyretin amyloid cardiomyopathy (ATTR-CM) develops when amyloid proteins collect and form deposits in the walls of the left ventricle. Theamyloid deposits cause the ventricle's walls to stiffen, which prevents the ventricle from filling with blood and reduces its ability to pump blood out of the heart. Myocardial fibrosis is defined as an increased quantity of collagenous scar tissue in the heart. Hypertrophic cardiomyopathy (HCM) is a disease in which the heart muscle becomes thickened (hypertrophied). The thickened heart muscle can make it harder for the heart to pump blood.

While there is shown and described herein certain specific structures embodying various embodiments of the invention, it will be manifest to those skilled in the art that various modifications and rearrangements of the parts may be made without departing from the spirit and scope of the underlying inventive concept and that the same is not limited to the particular forms herein shown and described except insofar as indicated by the scope of the appended claims.

What is claimed is:

1. An ultrasound imaging and backscatter system comprising:
an ultrasonic catheter having a longitudinal axis, a proximal end, and a distal end;
an ultrasonic pMUT transducer array disposed within the distal end of the ultrasonic catheter, wherein the ultrasonic pMUT transducer array comprises a substrate and a plurality of transducer array elements arranged on the substrate; and
a catheter shaft connected at one end to a handle assembly and at other end to the ultrasonic transducer array, wherein the catheter shaft houses an electronic flex cable which is in communication with at least one signal trace, and is configured to:
direct each of the plurality of transducer array elements, via the at least one signal trace, to transmit and receive, with respect to heart, ultrasound beams;
receive at least one signal from the plurality of transducer array elements based on transmitting and receiving at least one ultrasound beam of the ultrasound beams; and
construct at least one image of at least a portion of the heart based on the at least one signal.

2. The ultrasound imaging and backscatter system of claim 1, wherein the ultrasonic transducer array corresponds to a micro-electromechanical (MEMS) based Piezoelectric Micromachined Ultrasonic Transducer (pMUT).

3. A method of characterizing one or more tissue components of a scanned object in a patient, the method comprising:
positioning a catheter having a Piezoelectric Micromachined Ultrasonic Transducer Intracardiac echocardiographic (pMUT ICE) catheter adjacent to a region of interest of the scanned object;
receiving reflected signals from the pMUT ICE catheter;
scanning the region of interest;
determining one or more signal properties of the region of interest from the reflected signals;
associating the one or more signal properties to predetermined signal properties of tissue components of an object similar to the scanned object, wherein the predetermined signal properties comprise classification conditions stored in a data structure; and
identifying the one or more tissue components including tissue scaring, lesion assessment and measurement of tissue thickness.

4. The method of claim 3, where the identification of the one or more tissue components is identification of myocardial scarring.

5. The method of claim 4, where the myocardial scarring is the accumulation of fibrosis tissue.

6. The method of claim 4, where the myocardial scarring is from the formation of excess tissue.

7. The method of claim 3, where the lesion assessment is Endocarditis and an inflammation of the inner lining of the heart.

8. The method of claim 3, where the lesion assessment is of the septum or other heart chamber walls.

9. The method of claim 3, wherein the tissue thickness corresponds to the left ventricle, right ventricle, left atrium or right atrium thickness.

10. The method of claim 3, wherein the reflected signals include a plurality of backscattered scan lines and the determining step includes determining signal properties for a plurality of segments from the plurality of scan lines.

11. The method of claim 3, further comprising measuring wall structure used in conjunction with an ICE imaging ablation and mapping catheters.

12. A system comprising:
a catheter including a Piezoelectric Micromachined Ultrasonic Transducer Intracardiac echocardiographic (pMUT ICE) catheter configured to scan a region of interest;
a computing device in communication with the catheter, and configured to:
receive reflected signals from the pMUT ICE catheter;
determine one or more signal properties of the region of interest from the reflected signals;
associate the one or more signal properties to predetermined signal properties of one or more tissue components of an object similar to the scanned object, wherein the pre-determined signal properties comprise classification conditions stored in a data structure; and
identify the one or more tissue components including tissue scaring, lesion assessment and measurement of tissue thickness.

13. The system of claim 12, wherein the reflected signals include a plurality of backscattered scan lines, and the computing device is configured to determine signal properties for a plurality of segments from the plurality of backscattered scan lines.

14. An intracardiac echocardiographic (ICE) imaging and backscatter system comprising:
an ultrasonic catheter having a longitudinal axis, a proximal end, and a distal end;
a micro-electromechanical (MEMS) based Piezoelectric Micromachined Ultrasonic Transducer (pMUT) array disposed within the distal end of the ultrasonic catheter, wherein the MEMS based pMUT array comprises a substrate and a plurality of MEMS based pMUT array elements arranged on the substrate; and
an electronic flex cable connected at one end to a handle assembly and at other end to the MEMS based pMUT array, wherein the electronic flex cable is in communication with at least one signal trace, and is configured to:
direct each of the plurality of MEMS based pMUT array elements, via the at least one signal trace, to transmit and receive, with respect to heart, ultrasound beams;
receive at least one signal from the plurality of MEMS based pMUT array elements based on transmitting and receiving at least one ultrasound beam of the ultrasound beams; and
construct at least one image of at least a portion of the heart based on the at least one signal.

15. The ICE imaging and backscatter system of claim 14, wherein the MEMS based pMUT array comprises pMUT cells of multiple diameters to achieve a bandwidth of greater than 55%.
